(19) United States
(12) Patent Application Publication
(10) Pub. No.: US 2015/0196421 A1
(43) Pub. Date: Jul. 16, 2015

(54) Condom with Surrounding Prophylactic Shield

(71) Applicant: Josh Bolles, Minot, ND (US)

(72) Inventor: Josh Bolles, Minot, ND (US)

(21) Appl. No.: 14/587,428

(22) Filed: Dec. 31, 2014

Related U.S. Application Data
(60) Provisional application No. 61/927,534, filed on Jan. 15, 2014.

(51) Int. Cl. A61F 6/04

(52) U.S. Cl. CPC A61F 6/04

(57) ABSTRACT

A condom with a surrounding prophylactic shield provides a thick inner lining, preferably one-eighth inch thick, covers the user's penis. The inner lining is placed at the center of a circular shield manufactured from soft rubber or latex, preferably four inches in diameter. The shield is sufficiently deformable for sexual intercourse and ensures no skin to skin contact between the participants, thus preventing the spread of contact STDs.

### CROSS-REFERENCE TO RELATED APPLICATIONS

This application claims the benefit of U.S. Provisional Application No. 61/927534, filed Jan. 15, 2014.

### STATEMENT REGARDING FEDERALLY SPONSORED RESEARCH OR DEVELOPMENT

Not Applicable

### PARTIES TO A JOINT RESEARCH AGREEMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The invention relates generally to prophylactic devices and in particular to a condom with a surrounding prophylactic shield. Sexually transmitted diseases (STDs) are the plague of the 21st century. The disease prevention function of a condom is well-known, but is limited to preventing the exchange of bodily fluids. Skin to skin contact in the pubic area is unprotected, and the transmission of both diseases and parasites may occur. A condom with a surrounding prophylactic shield would resolve this problem. A thick inner lining, preferably one-eighth inch thick, covers the user's penis. The inner lining is placed at the center of a circular shield manufactured from soft rubber or latex, preferably four inches in diameter. The shield is sufficiently deformable for sexual intercourse and ensures no skin to skin contact between the participants, thus preventing the spread of contact STDs.

### SUMMARY OF THE INVENTION

Accordingly, the invention is directed to a condom with a surrounding prophylactic shield. A thick inner lining, preferably one-eighth inch thick, covers the user's penis. The inner lining is placed at the center of a circular shield manufactured from soft rubber or latex, preferably four inches in diameter. The shield is sufficiently deformable for sexual intercourse and ensures no skin to skin contact between the participants, thus preventing the spread of contact STDs.

Additional features and advantages of the invention will be set forth in the description which follows, and will be apparent from the description, or may be learned by practice of the invention. The foregoing general description and the following detailed description are exemplary and explanatory and are intended to provide further explanation of the invention.

### BRIEF DESCRIPTION OF THE DRAWINGS

The accompanying drawings are included to provide a further understanding of the invention and are incorporated into and constitute a part of the specification. They illustrate one embodiment of the invention and, together with the description, serve to explain the principles of the invention.

FIG. 1 is a side perspective view of the first exemplary embodiment, displaying the inner lining 10, the base of the inner lining 10A, and the shield 11.

FIG. 2 is a side view of the first exemplary embodiment, displaying the inner lining 10, the base of the inner lining 10A, and the shield 11.

FIG. 3 is a rear view of the first exemplary embodiment, displaying the inner lining 10, the base of the inner lining 10A, and the shield 11.

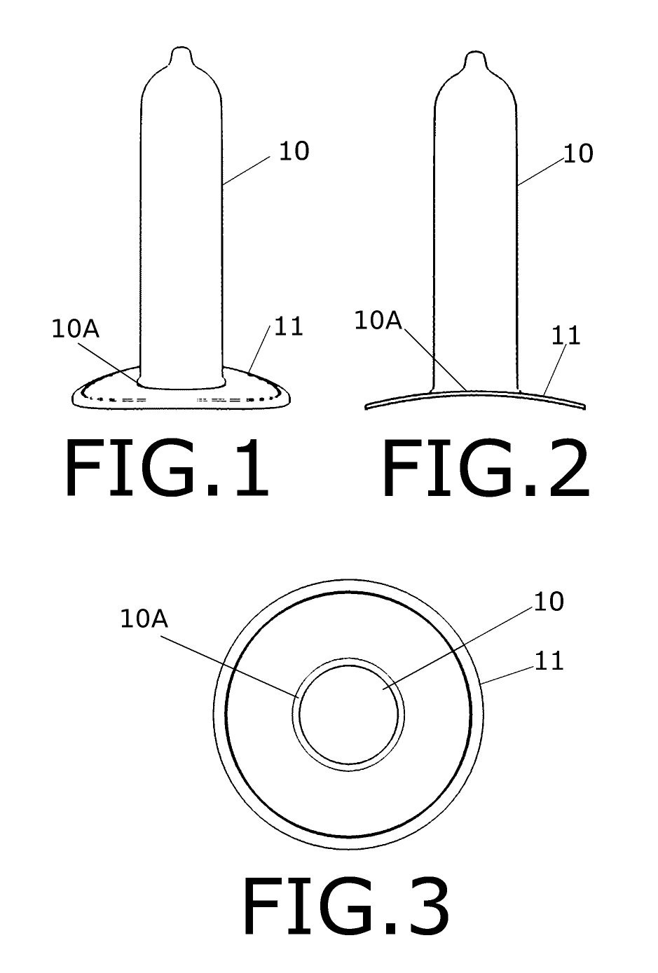

### DETAILED DESCRIPTION OF THE INVENTION

Referring now to the invention in more detail, the invention is directed to a condom with a surrounding prophylactic shield 11. A thick inner lining 10, preferably one-eighth inch thick, covers the user's penis. The shape of the inner lining may be generally understood as condom-shaped, being a cylinder closed at the tip, optionally with a semen reservoir per standard condom design, as shown, and open at the base 10A. The inner lining 10 is circular at its base 10A, which is circumferentially attached to at the center of a circular shield 11 manufactured from soft rubber or latex, preferably four inches in diameter, with both the shield 11 and base 10A having an coextensive annular opening through which the user's penis is inserted. The shield 11 is sufficiently deformable for sexual intercourse and ensures no skin to skin contact between the participants (a male human user and a partner; skin to skin contact being understood as at least contact between the male user's genital region and any part of the partner's body coming into mechanical contact therewith), thus preventing the spread of contact STDs.

The first exemplary embodiment features an inner lining 10, preferably one-eighth inch thick, which covers the user's penis. Thinner and thicker inner linings 10 are also contemplated, for example, standard condom thicknesses of less than 1 millimeter are contemplated. The inner lining 10 is located at the center of a circular shield 11, preferably four inches in diameter. The shield 11 is outwardly convex (in the direction of the user's penis), with an inward curvature which accommodates the shape of the male pubic area. Shields 11 of different shapes and sizes are also contemplated.

To use the first exemplary embodiment, the user inserts the penis into the inner ling 10, such that the concave surface of the shield 11 rests against the pubic area of the user's body. The user may then safely engage in sexual activity.

The inner lining 10 and the shield 11 are preferably manufactured from a flexible, durable material such as latex, rubber, or silicone. Components, component sizes, and materials listed above are preferable, but artisans will recognize that alternate components and materials could be selected without altering the scope of the invention.

While the foregoing written description of the invention enables one of ordinary skill to make and use what is presently considered to be the best mode thereof, those of ordinary skill in the art will understand and appreciate the existence of variations, combinations, and equivalents of the specific embodiment, method, and examples herein. The invention should, therefore, not be limited by the above described embodiment, method, and examples, but by all embodiments and methods within the scope and spirit of the invention.

I claim:

1. A condom with prophylactic shield comprising a lining; said lining being condom-shaped; said lining having a base; said lining being open at said base; said base being affixed circumferentially to a shield.

2. The condom with prophylactic shield of claim 1 wherein said lining is one eighth inch thick.

3. The condom with prophylactic shield of claim 1 wherein said shield is four inches in diameter.

4. The condom with prophylactic shield of claim 2 wherein said shield is four inches in diameter.

5. The condom with prophylactic shield of claim 1 wherein said shield is of a convex shape.

6. The condom with prophylactic shield of claim 2 wherein said shield is of a convex shape.

7. The condom with prophylactic shield of claim 3 wherein said shield is of a convex shape.

8. The condom with prophylactic shield of claim 4 wherein said shield is of a convex shape.

9. A method of using a condom with prophylactic shield, the condom with prophylactic shield comprising a lining; said lining being condom-shaped; said lining having a base; said lining being open at said base; said base being affixed circumferentially to a shield; and the method comprising a human male user inserting his penis into said lining via said base such that said shield covers his pubic area; and proceeding to engage in sexual intercourse with a partner; whereby said shield prevents skin to skin contact between said male human user and said partner.

10. The method of claim 9 wherein said lining is one eighth inch thick.

11. The method of claim 9 wherein said shield is four inches in diameter.

12. The method of claim 10 wherein said shield is four inches in diameter.

13. The method of claim 9 wherein said shield is of a convex shape.

14. The method claim 10 wherein said shield is of a convex shape.

15. The method of claim 11 wherein said shield is of a convex shape.

16. The method of claim 12 wherein said shield is of a convex shape.
